(19) United States
(12) Patent Application Publication
(10) Pub. No.: US 2022/0061899 A1
(43) Pub. Date: Mar. 3, 2022

(54) Method For Generating Digital Models Of Osteosynthesis Plates Specific To The Patient's Morphology

(71) Applicant: Abys Medical, La Rochelle (FR)

(72) Inventors: Arnaud DESTAINVILLE, Saint-Xandre (FR); Olivier RICHART, Le Bois-Plage-En-Re (FR)

(73) Assignee: Abys Medical, La Rochelle (FR)

(21) Appl. No.: 17/408,969

(22) Filed: Aug. 23, 2021

Related U.S. Application Data
(60) Provisional application No. 63/073,559, filed on Sep. 2, 2020.

(51) Int. Cl. A61B 17/80; A61B 34/10; B33Y 80/00

(52) U.S. Cl. CPC A61B 17/8052; A61B 34/10; A61B 2017/00526; A61B 2034/108; B33Y 80/00

(57) ABSTRACT

A method of generating an osteosynthesis plate including obtaining a bone model representing a bone generated from imaging data corresponding to the bone, determining a set of virtual entry locations on the bone model, and constructing a virtual surface adjacent the bone model based on the bone model and the set of virtual entry locations.

### CROSS-REFERENCE TO RELATED APPLICATION

The application claims priority to U.S. provisional patent application Ser. No. 63/073,559 filed on Sep. 2, 2020, which is incorporated by reference herein.

### FIELD

The present disclosure relates to a method for manufacturing an osteosynthesis plate as well as to osteosynthesis plates manufactured in this way.

### BACKGROUND

Users or patients receiving care from a surgeon or operator are often times treated using one-size-fits-all mentality, especially with respect to commonly performed surgeries. Such a mentality extends to the actual implants or devices used for each patient, even though each patient is different. This is in part due to it being impractical to generate customized materials for each patient due to cost, time, and technology limitations.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A method of generating an osteosynthesis plate including obtaining a bone model representing a bone generated from imaging data corresponding to the bone, determining a set of virtual entry locations on the bone model, and constructing a virtual surface adjacent the bone model based on the bone model and the set of virtual entry locations. The method includes generating an osteosynthesis plate model based on the virtual surface, creating a virtual hole at each virtual entry location of the set of virtual entry locations along the osteosynthesis plate model, and transmitting the osteosynthesis plate model to an osteosynthesis plate-manufacturing machine.

In other aspects, the set of virtual entry locations indicate locations where a screw is inserted into the bone. In other aspects, the bone model is three-dimensional. In other aspects, the method includes obtaining the imaging data corresponding to the bone from an imaging device. In other aspects, the virtual surface includes the set of virtual entry locations, a screw is one of multiple screws and the virtual hole is one of multiple holes. In other aspects, the osteosynthesis plate model includes a virtual face defined by the virtual surface and a thickness defined by an extrusion of the virtual face.

In other aspects, the method includes generating a virtual scene including the bone model. In other aspects, the osteosynthesis plate model is pressed against the bone model and positioned such that a virtual face of the osteosynthesis plate model coincides with the virtual surface. In other aspects, the osteosynthesis plate model generation and subsequent manufacturing of a custom osteosynthesis plate prevents a need for a surgeon to modify the osteosynthesis plate during surgery.

In other aspects, the virtual surface is electronically determined from a virtual generatrix curve that passes successively through reference points, the reference points being determined as centers of the set of virtual entry locations and of at least two lateral virtual curves that are arranged on either side of the virtual generatrix curve.

In other aspects, the reference points include longitudinal ends, with the virtual generatrix curve passing successively through one of the longitudinal ends, centers of the set of virtual entry locations, and another of the longitudinal ends. In other aspects, the virtual generatrix curve is a geodesic-type spline passing through the reference points.

In other aspects, the at least two lateral virtual curves are geodesic-type splines that pass through images of the reference points. In other aspects, the images are obtained through translation of a distance that is a function of a width of a screw at a greatest diameter perpendicular to an axis of the osteosynthesis plate model and tangent to the virtual surface of the bone model. In other aspects, the at least two lateral virtual curves are virtual curves that are that are spaced apart on each side of the virtual generatrix curve by a predetermined distance and projected on the virtual bone surface.

In other aspects, the method includes determining an intermediate osteosynthesis plate model by adding virtual fillets to the osteosynthesis plate model at longitudinal ends of the osteosynthesis plate model. In other aspects, the determination of the osteosynthesis plate model is performed by modifying the intermediate osteosynthesis plate model. In other aspects, the osteosynthesis plate-manufacturing machine is an additive or subtractive manufacturing machine.

In other aspects, the osteosynthesis plate that is manufactured is made of medical-grade materials. In other aspects, the osteosynthesis plate-manufacturing machine uses laser sintering or three-dimensional printing and is at a remote location from the modelling.

Computer software includes instructions, stored in a non-transitory computer-readable memory. The software instructions include receiving a bone model of a bone generated from data representing the bone and obtaining virtual entry locations on the bone model. The software instructions include modelling an osteosynthesis plate model by determining a virtual surface on a face of the bone model based on the bone model and the virtual entry locations and drilling a virtual hole designed to receive a virtual screw at each virtual entry location along the osteosynthesis plate model. The software instructions include transmitting the osteosynthesis plate model to a remote osteosynthesis plate-manufacturer.

In other aspects, the osteosynthesis plate model includes a virtual face defined by the virtual surface and a thickness defined by an extrusion of the virtual face.

In other aspects, the instructions include generating a virtual scene including the bone model. In other aspects, the osteosynthesis plate model is pressed against the bone model and positioned such that a virtual face of the osteosynthesis plate model coincides with the virtual surface.

In other aspects, the virtual surface is determined from a virtual generatrix curve that passes successively through reference points, the reference points being determined as centers of the virtual entry locations and of at least two lateral virtual curves that are arranged on either side of the virtual generatrix curve.

In other aspects, the reference points include longitudinal ends, with the virtual generatrix curve passing successively through one of the longitudinal ends, centers of the virtual entry locations, and another of the longitudinal ends.

A method of generating a surgical model including obtaining imaging data, generating a bone model based on the imaging data of a bone, and constructing a virtual surface on a face the bone model using the bone model. The method includes receiving a drill position on the bone model, generating a surgical model based on the virtual surface and the drill position, and sending the surgical model to a surgical model-manufacturing machine.

In other aspects, the surgical model is an osteosynthesis plate model, a surgical guide, or both an osteosynthesis plate model and a surgical guide. In other aspects, the surgical model-manufacturing machine is an additive or subtractive manufacturing machine. In other aspects, the surgical model that is manufactured is made of medical-grade materials.

A model generation system including a graphical user interface, at least one processor, and a memory coupled to the at least one processor. The memory stores instructions executed by the at least one processor. The instructions include retrieving a bone model from imaging data corresponding to a bone, displaying, via the display, the bone model, and receiving input, via the display, indicating a virtual entry location on the bone model. The instructions include constructing a virtual surface adjacent the bone model based on the bone model and the virtual entry location, creating an osteosynthesis plate model based on the virtual surface, and drilling a virtual hole at the virtual entry location on the osteosynthesis plate model. The instructions include transmitting the osteosynthesis plate model to a plate model storage.

In other aspects, the osteosynthesis plate model includes a virtual face defined by the virtual surface and a thickness defined by an extrusion of the virtual face.

In other aspects, the instructions include generating a virtual scene including the bone model. In other aspects, the osteosynthesis plate model is pressed against the bone model and positioned such that a virtual face of the osteosynthesis plate model coincides with the virtual surface. In other aspects, the osteosynthesis plate model generation and subsequent manufacturing of a custom osteosynthesis plate prevents a need for a surgeon to modify the plate during surgery.

In other aspects, the virtual surface is electronically determined from a virtual generatrix curve that passes successively through reference points, the reference points being determined as centers of the drill position and of at least two lateral virtual curves that are arranged on either side of the virtual generatrix curve.

In other aspects, the reference points include longitudinal ends, with the virtual generatrix curve passing successively through one of the longitudinal ends, centers of the drill position, and another of the longitudinal ends.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a perspective view of a representation of a three-dimensional bone model in which virtual screws are positioned.

FIG. 2 is a view similar to FIG. 1, the virtual screws being removed from the bone, leaving only a trace of virtual entries of the virtual screws on the surface of the bone.

FIG. 3 is a view similar to FIG. 2, further comprising longitudinal ends.

FIG. 4 is a view similar to FIG. 3, further comprising a generatrix curve that passes through reference points.

FIG. 5 is a view similar to FIG. 4, further comprising at least two lateral curves that frame the generatrix curve.

FIG. 6 is a view similar to FIG. 5, comprising a virtual surface that is determined by the at least two lateral curves of FIG. 4.

FIGS. 7A-7C are similar to FIG. 1, comprising a three-dimensional model of bone and a model of an osteosynthesis plate.

FIGS. 8A-8B includes three-dimensional models of bone and a model of a perforated osteosynthesis plate.

FIG. 9 shows a three-dimensional model of a perforated osteosynthesis plate.

FIG. 10 is a high-level block diagram of a plate generation system according to the principles of the present disclosure.

FIG. 11 is an example additive manufacturing machine creating a perforated osteosynthesis plate.

FIG. 12 is a flowchart describing generation of a perforated osteosynthesis plate model.

In the drawings, reference numbers may be reused to identify similar and/or identical elements.

### DETAILED DESCRIPTION

### Technical Problems Addressed by the Present Disclosure

An osteosynthesis plate, sometimes also referred to as a “bone plate” in the literature, is generally used to keep different parts of a fractured or otherwise severed bone substantially stationary relative to one another during and/or after the consolidation process, during which the bone repairs itself. Fractures of a bone in the region of the head can be particularly troublesome due to movement and/or the presence of soft tissue in the osteoarticular region.

Typically, an orthopedic osteosynthesis plate can comprise an elongate portion that can be fixed to a body of bone using a plurality of bone-anchored screws, said elongated portion defining a longitudinal axis, a flared portion that is capable of being fixed to a head of the bone using at least one of the bone screws, and an intermediate portion that interconnects said elongate portion and said flared portion.

A plurality of sizes of plates can be provided having different widths and lengths. However, they are not fitted exactly to each anatomy. The surgeon will often use a straight plate and deform it. The plate can have a basic, roughly anatomical shape with a curvature, but this curvature is not adapted to each morphology.

After all, substantial anatomical variability exists in the inter- and intrapopulational skeleton, requiring wide dimensional intervals to be defined as well as dedicated designs for retention systems in order to address all of the situations that are encountered by practitioners. It is also proposed that the surgeon himself perform the anatomical deformation of the proposed plates in order to optimize contact with the bone. This practice is not very satisfactory, however, because it is often imprecise and time-consuming and thus constitutes a risk situation during a surgical intervention. This practice also requires the practitioner to use massive forceps to deform metallic material, which may be damaged when subjected to such stresses, portending a potential risk for the postoperative period.

The variability of designs must also include the great number of types of skeletal fracture identified (long bones, spinal bones, simple or compound fracture, extra-articular or intra-articular fracture, transverse fracture, multi-fragmentary fracture, etc.), in addition to osteotomies—which, by nature, occur intentionally. For long bone fractures alone, the classification carried out by Müller identifies more than 120 different fractures, at least 50 of which are conventionally treated by osteosynthesis.

The consequence of all of these requirements, increased by the variety of possible manufacturing materials, is that a plethora of osteosynthesis devices are being made available to orthopedic surgeons and human or veterinary traumatologists. It is therefore not uncommon for a practitioner to have to choose during the intervention from among several dozen plates and screws in order to carry out his intervention, these plates and screws being associated with several instruments that are specially designed for the placement of the associated implants. In addition to the time that the practitioner must take refining his preoperative analysis in order to determine the products that he will be requiring among the dozens offered, it is necessary to integrate the cumbersome management of this stock, both upstream and downstream, whether in terms of the financial and administrative impact or the management of the cleaning and sterilization of these devices. In view of the historical choice from among the overabundance of products aimed at meeting all of the needs of orthopedic and traumatological practitioners, the operational impact for healthcare establishments is inseparable from the analysis.

The manufacture of tailor-made osteosynthesis systems, and more particularly of plates, thus represents one solution for better meeting the expectations of practitioners on the surgical level, but—by extension, and through an adapted and competitive manufacturing process and simple and attractive surgical instruments—it must allow for integration into a broader offering that will also have to optimize the operational impact within healthcare establishments and medical teams. The present disclosure provides a solution to these problems by providing a method for generating digital files for plates that are specific to the anatomy of the patient for the purpose of manufacturing them.

Today, it is possible to plan an intervention based on 3D representations of the bone to be treated. These 3D representations can be obtained, for example, from scanographic data obtained by tomodensitometry or even magnetic resonance imaging (MRI). This planning is generally accompanied by a simulation of the different stages of the intervention, particularly the position and angles of different cutting planes (in the case of osteotomy to correct deformed bones) or the repositioning of bone fragments relative to others (in the case of fractures).

In order to help the surgeon to adhere as closely as possible to the pre-established surgical plan in the case of a corrective osteotomy, for example, dedicated and personalized surgical guides are sometimes made available to practitioners. These cutting guides have bone attachment surfaces that match the shape of the latter, which makes it possible to position the cutting planes and the holes in a precise and unambiguous manner. However, the prior art does not make it possible to offer bone retention (osteosynthesis) plates that are custom-designed prior to their manufacture.

### Introduction

According to a first aspect of the present disclosure, a method is proposed for generating an osteosynthesis plate that is suitable for being fixed to a bone, comprising a step of obtaining a three-dimensional bone model representing the bone from an imaging device for imaging said bone, three-dimensional data for the bone and, on the basis of the three-dimensional data obtained, a step of receiving virtual entry locations of a plurality of virtual anchoring objects from a three-dimensional representation that includes a representation of a virtual bone generated from the three-dimensional bone model to which a plurality of virtual anchoring objects are added, and a step of generating a virtual surface disposed on the virtual bone, the virtual surface being generated from the virtual entry locations of the plurality of virtual anchoring objects, said virtual surface comprising the virtual entry locations.

The method includes a step of generating a model of an osteosynthesis plate, the osteosynthesis plate having a virtual face that is defined by the virtual surface and a thickness that is obtained through extrusion of the virtual face, a step of generating a virtual scene comprising the virtual bone, the virtual osteosynthesis plate model being pressed against the virtual bone and positioned such that its virtual face coincides with the virtual surface, a step of determining a second model of the osteosynthesis plate by modifying the model of the osteosynthesis plate by virtual drilling of the osteosynthesis plate over its thickness to form holes in virtual anchors that are designed to receive the plurality of virtual anchoring objects, and a step of sending the modified osteosynthesis plate model to a device that makes it possible to manufacture an osteosynthesis plate from the modified osteosynthesis plate model.

The imaging device can be a computed tomography scanner or an MRI, for example. The three-dimensional data for the bone can be reconstructed, for example, from files in DICOM format (Digital Imaging and Communications in Medicine), which is a standard for the IT management of data from medical imaging. The virtual anchoring objects can be virtual screws, for example. Alternatively, or in addition, anchoring objects can be pins or any other type of implant that can be attached to the plate.

Virtual anchoring objects such as virtual screws are solid polygon-based elements. Typically, such virtual anchoring objects are formed from a plurality of meshes. A mesh is a three-dimensional object made up of vertices, edges, and faces that are organized into polygons in the form of a wire-frame in a three-dimensional infographic. The faces are usually composed of triangles, quadrilaterals, or other simple convex polygons, as this simplifies rendering. The faces can be combined to form more complex concave polygons or polygons with holes. The virtual anchoring objects can be of different dimensions in terms of length or diameter. The virtual anchoring screws can have different types of imprint, and possibly several levels of imprints that possibly have a plurality of types of imprint. The virtual screws can be cortical or spongy, and locked or not.

The virtual entry locations of the virtual anchoring objects can be determined by a user, for example. To this end, the user can specify these entry locations on a representation of the bone model. According to a first option, the virtual exit locations of the virtual anchoring objects can be determined by the user. To this end, the user can specify these exit locations on a representation of the bone model.

According to a second option, the orientation of the virtual anchoring objects can be determined in such a way that, once positioned at the virtual entry location according to said orientation, the virtual anchoring object is directed toward the center of the medullary canal of the bone. In other words, the virtual anchoring object is directed toward the barycenter of the medullary canal of the bone perpendicular to the longitudinal axis that passes through the virtual entry location.

According to one variant, the virtual anchoring objects can be positioned based on processing carried out by a computing unit on the basis of the three-dimensional bone model. In addition, the positioning of the virtual anchoring objects positioned by the computing unit can be validated—or modified—by a surgeon who alone has the qualification to perform a movement that may affect the therapeutic choice. The virtual entry locations of the plurality of virtual anchoring objects are preferably disposed within the topological interior of the virtual surface. The present description is aimed at the method for obtaining a three-dimensional object from a three-dimensional surface through extrusion of the virtual face. This is common terminology in the field of vector images, for example.

According to one option, the virtual surface is determined from a virtual generatrix curve that passes successively through reference points, the reference points being determined as the centers of the virtual entry locations and of at least two lateral virtual curves that are arranged on the bones on either side of the virtual curve.

The method according to the present disclosure can also include positioning of longitudinal ends of a virtual plate model on the surface of the virtual three-dimensional bone model, said longitudinal ends being determined from the entry locations of the plurality of virtual anchoring objects and positioned on the surface of the three-dimensional bone model.

When the longitudinal ends are positioned on the surface of the three-dimensional bone model, the method according to the present disclosure can further comprise a step of determining an intermediate osteosynthesis plate model by adding virtual fillets to the osteosynthesis plate model at the longitudinal ends, the determination of the second osteosynthesis plate model being performed by modifying said intermediate osteosynthesis plate model.

When longitudinal ends are positioned on the surface of the three-dimensional bone model, the reference points also include the longitudinal ends, with the virtual generatrix curve passing successively through one of the longitudinal ends, the centers of the virtual entry locations, and another of the longitudinal ends. The virtual curve can be a spline representing a geodesic that passes through the reference points on the surface of the virtual bone.

According to a first option, the at least two lateral virtual curves can be splines that pass through images of the reference points, said images being obtained through translation of a distance that is a function of the width of the screw at the greatest diameter perpendicular to the axis of the plate and tangent to the surface of the bone. Also, the image points are tangent to the surface of the bone and are offset by half a plate width perpendicular to the spline.

According to another option that can be optionally combined with the first, the at least two lateral virtual curves are virtual curves that run that are spaced apart on each side of the virtual generatrix curve by a predetermined distance and projected on the virtual bone surface.

During the step of determining the second model of the osteosynthesis plate, the virtual drilling step can be carried out by subtracting models of holes, which advantageously depend on the type and size of the virtual anchoring object, of the osteosynthesis plate over its thickness to form holes in virtual anchoring objects that are designed to receive the plurality of virtual anchoring objects.

The device that makes it possible to manufacture an osteosynthesis plate from the modified osteosynthesis plate model can be of the additive manufacturing machine type, often referred to by the generic term “3D printer.” However, it is possible to use traditional manufacturing methods such as multi-axis machining or injection and rework molding for the production of the bores in the plate manufactured in this way. The manufactured osteosynthesis plate can be made of medical-grade metallic material. In particular, this type of material includes 316L stainless steel, pure titanium, or even TA6V or TA6V-Eli titanium alloy.

According to a second aspect of the present disclosure, a device is proposed for generating an osteosynthesis plate that is suitable for being fixed to a bone comprising a computing unit that is configured for: obtaining a three-dimensional bone model representing the bone on the basis of three-dimensional data obtained from an imaging device enabling said bone to be imaged, obtaining virtual entry locations of a plurality of virtual anchoring objects from a representation comprising a three-dimensional representation of a virtual bone generated from the three-dimensional bone model to which a plurality of positioned virtual anchoring objects are added, with each of the virtual anchoring objects being positioned between a virtual entry location on the virtual bone and a virtual exit location on the virtual bone, and generating a virtual surface that is arranged on the virtual bone, the virtual surface being generated from the virtual entry locations of the plurality of virtual anchoring objects, said virtual surface comprising the virtual entry locations.

The computing unit that is configured for generating a model of an osteosynthesis plate, the osteosynthesis plate model having a virtual face that is defined by the virtual surface and a thickness that is obtained by extrusion of the virtual face, generating a virtual scene comprising the virtual bone, the virtual osteosynthesis plate model being pressed against the virtual bone and positioned such that its virtual face coincides with the virtual surface, determining a model of a perforated osteosynthesis plate by modifying the model of the osteosynthesis plate by virtual drilling of the osteosynthesis plate over its thickness to form holes for virtual anchoring objects designed to receive the plurality of virtual anchoring objects, and sending the perforated osteosynthesis plate model to a device that makes it possible to manufacture an osteosynthesis plate from the modified osteosynthesis plate model.

According to a second aspect of the present disclosure, a system is provided for generating an osteosynthesis plate that is suitable for being fixed to a bone, comprising a device for generating an osteosynthesis plate according to the first aspect of the present disclosure, or one or more improvements thereof, and a device that makes it possible to manufacture an osteosynthesis plate from the model of the perforated osteosynthesis plate received from said generation device.

### Bone Model

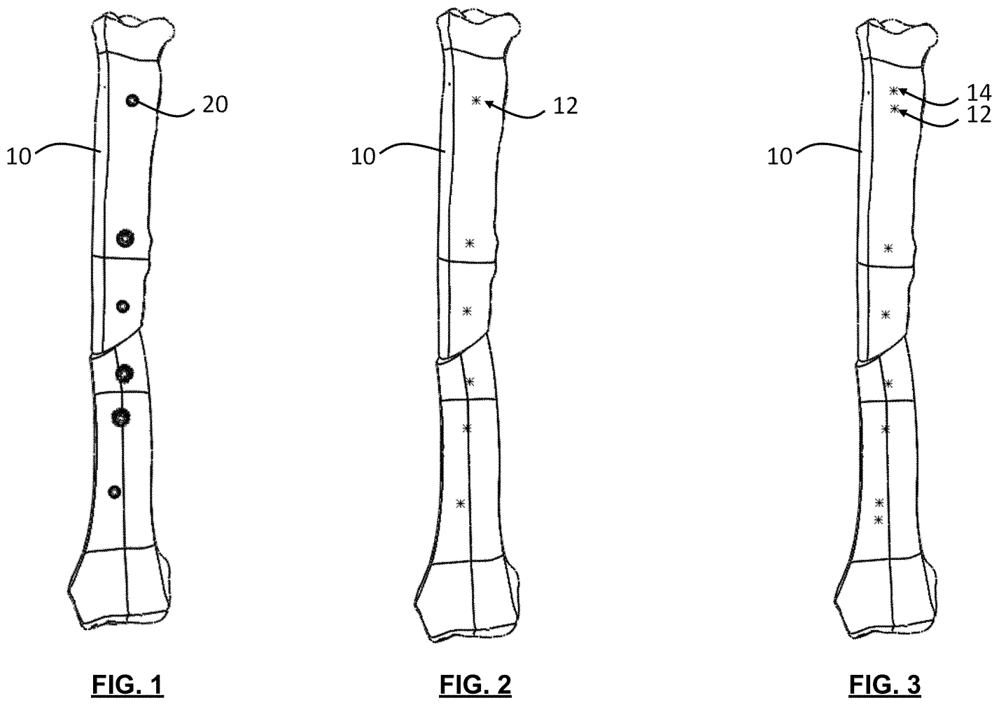

Referring to FIG. 1, one sees a perspective view of a representation of a bone model with positioned screws 1. In the following description, only one type of virtual anchor is given, namely that of the virtual screw. The bone model with positioned screws comprises a bone model 10 on the one hand and a screw 20 on the other hand. The screw 20 is positioned in the bone.

The three-dimensional bone model 10 is obtained from three-dimensional data relating to real bone that are obtained from an imaging device. The three-dimensional data can be in a DICOM format, for example. The three-dimensional data can be sent to the computing unit, which is configured to determine the three-dimensional bone model 10 from said three-dimensional data.

In order to obtain the bone model with positioned screw 1, a representation of the bone model 10 can be presented to a user—for example on a display screen such as a computer screen, or even by means of a virtual reality or hologram generation device (augmented or mixed reality). The user can select one screw or a plurality of screws and position it or them on the representation, and hence in the bone model, thus generating the three-dimensional bone model with positioned screws 1.

To position the screw, the user may be given the option to indicate an entry location for a screw on the representation of the bone model. For example, the user can indicate the entry location by means of a pointing device, such as a mouse, and by pointing to the entry location of the screw on the representation of the bone model. The entry locations can be sent to the computing unit.

According to one option, an orientation of the screw is then determined in such a way that, once positioned at the entry location according to said orientation, the virtual screw is directed toward the center of the medullary canal of the bone.

Alternatively, the orientation of the virtual screw is determined by the user. The user may be given the option to indicate an exit location for the screw on the representation of the bone model. For example, the user can indicate the exit location by means of a pointing device, such as a mouse, and by pointing to the exit location of the screw on the representation of the bone model.

According to yet another option, the orientation of the virtual screw is determined by the user. The user may be given the option to indicate an exit location for the screw on the representation of the bone model. For example, the user can indicate the exit location by means of a pointing device, such as a mouse, and by moving the exit location on the model, for example by moving the tip of the screw on the representation of the bone model.

FIG. 2 is a perspective view of a representation of a bone model with positioned entry location points 2. The entry location bone model comprises the bone model 10 on the one hand and one or more entry locations 12 on the other hand. A plurality of entry locations are shown in FIG. 2. Also, the central unit can receive a three-dimensional model 10 and virtual entry locations of a plurality of virtual screws.

FIG. 3 is a perspective view of a representation of a bone model with entry locations and positioned ends 3. The bone model with entry location, for its part, comprises the bone model 10, an entry location 12, and a longitudinal end location 14. Two longitudinal end locations are shown in FIG. 3. In one variant of the present disclosure, the locations of longitudinal ends can be determined by the user.

Alternatively, the longitudinal end locations can be determined by the computing unit from the entry locations of the plurality of virtual screws and positioned on the surface of the bone model. The location of the distal longitudinal end is positioned on the surface of the bone in the extension of the two entry locations of the most distal virtual screws at a distance that depends on the size of the largest screw used. The same is true for the location of the proximal longitudinal end.

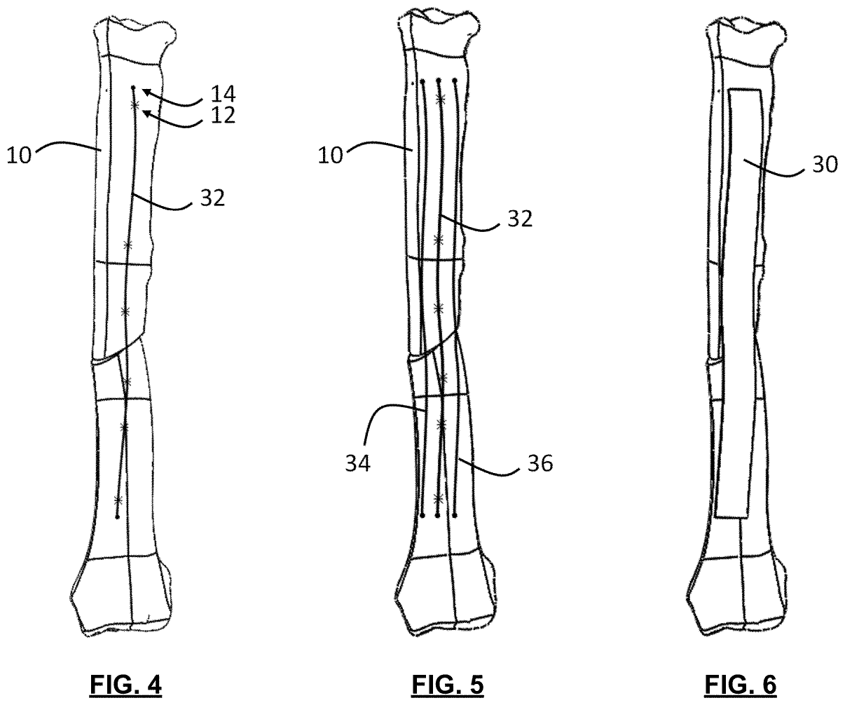

FIG. 4 is a perspective view of a representation of a bone model with virtual generatrix curve 4. The bone model with virtual generatrix curve 4, for its part, comprises the bone model 10, the entry location 12, the longitudinal end location 14, and a virtual generatrix curve 32. The virtual generatrix curve 32 passes successively through reference points. The reference points comprise the centers of the virtual entry locations 12. In the example shown, the reference points also include the longitudinal ends 14. The virtual generatrix curve passes successively through one of the longitudinal ends 14, the centers of the virtual entry locations 12, and another of the longitudinal ends 14. The generatrix curve can be determined in the manner of a spline. In this case, the reference points are called intermediate points.

In this case, the degree of the spline is typically 3. As will readily be understood, other degrees can be chosen. This parameter is an input parameter for the device according to the present disclosure. Of course, the spline is determined on the surface having the reference points. The spline passes through a finite number of points on the surface of the bone. It passes through anchor points and points defined on the bone surface so that it follows the shape of the bone, for example with points every 0.5 mm.

The spline is a geodesic-like curve that passes through intermediate points on the surface of the bone. The virtual generatrix curve is determined by the computing unit from the entry locations of the plurality of virtual screws and the longitudinal ends.

FIG. 5 is a perspective view of a representation of a bone model with virtual curves 5. The bone model with virtual generatrix curve 5, for its part, comprises the bone model 10, the virtual generatrix curve 32, and two lateral curves 34, respectively 36. According to one option, the lateral curves 34, respectively 36, are spaced apart on each side of the virtual generatrix curve by a predetermined distance and projected on the virtual bone surface. Typically, a distance can be a multiple of the diameter of the screws, for example three screw diameters.

FIG. 6 is a perspective view of a representation of a bone model with virtual surface 6. The bone model with virtual generatrix curve 6, for its part, comprises the bone model 10 and a virtual surface 30. The virtual surface is determined by the computing unit from the three virtual curves 32, 34, and 36.

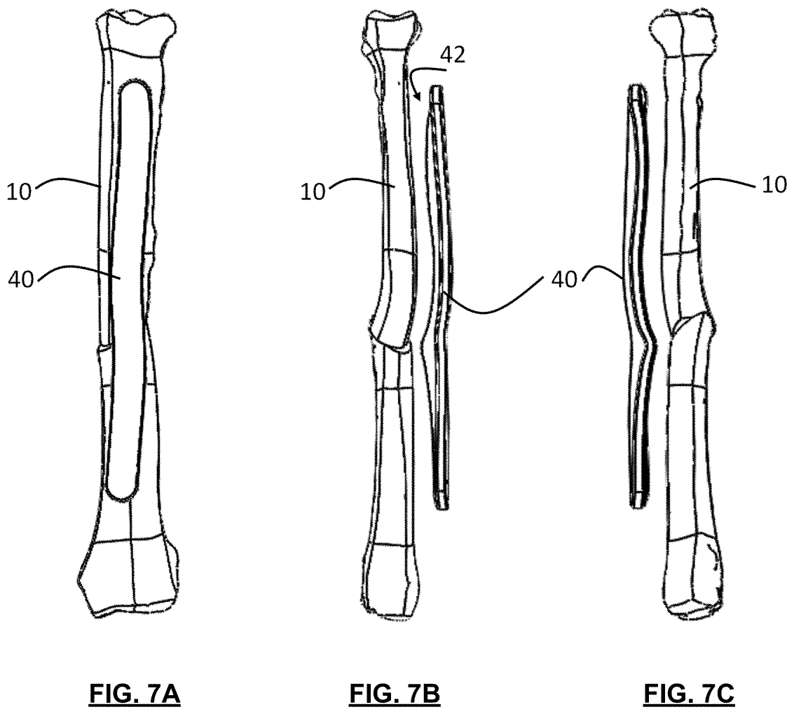

FIGS. 7A-7C illustrates a bone model with osteosynthesis plate 7. The bone model with osteosynthesis surface 7 comprises the bone model 10 on the one hand and a model 40 of the osteosynthesis plate on the other hand. The bone models 10 and the osteosynthesis plate model 40 are seen from the front in FIGS. 7A-7C. The model 40 of the osteosynthesis plate has a virtual face 42 that is defined by the virtual surface 30. When the model 40 is positioned on the bone model, the virtual face 42 and the virtual surface 30 coincide.

The model 40 is obtained by volumization of the virtual surface 42. For example, the model 40 is obtained by extruding the virtual face 42. The extrusion of a surface is aimed at achieving the method that enables a volume to be obtained by translation of the surface along a predetermined axis—for example, an axis passing through the center of the medullary canal of the bone. According to another option, the model 40 is obtained by filling between the virtual surface 42 and the surface parallel to 42 spaced apart by a predetermined distance corresponding to the thickness of the model 40. In addition, the computing unit can determine fillets at the longitudinal ends of the virtual surface 30.

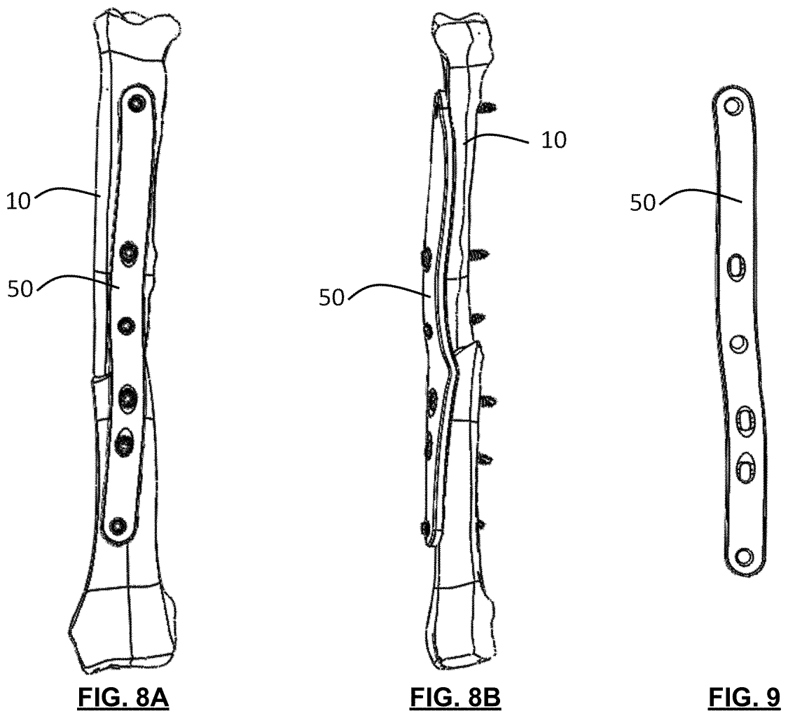

FIGS. 8A-8B illustrates a bone model with a perforated osteosynthesis plate and positioned screws 8. The bone model with virtual surface and positioned screws 8 comprises the bone model 10 on the one hand and a model 42 of a perforated osteosynthesis plate on the other hand. It also includes screws positioned in the model 50 and in the bone model 10. The bone model with virtual surface and positioned screws 8 is seen from the front in FIGS. 8A-8B.

To generate the perforated osteosynthesis plate model, the computing unit is configured to generate a representation comprising the bone model and the virtual osteosynthesis plate model 40 pressed against the virtual bone and positioned such that its virtual face coincides with the virtual surface 30.

The model 42 of the perforated osteosynthesis plate is obtained by modifying the model 40 of the osteosynthesis plate by virtual drilling of the osteosynthesis plate over its thickness to form virtual screw holes designed to receive the plurality of virtual screws. The perforated osteosynthesis plate model 40 can be of the volumetric file model type, for example in STL or OBJ format.

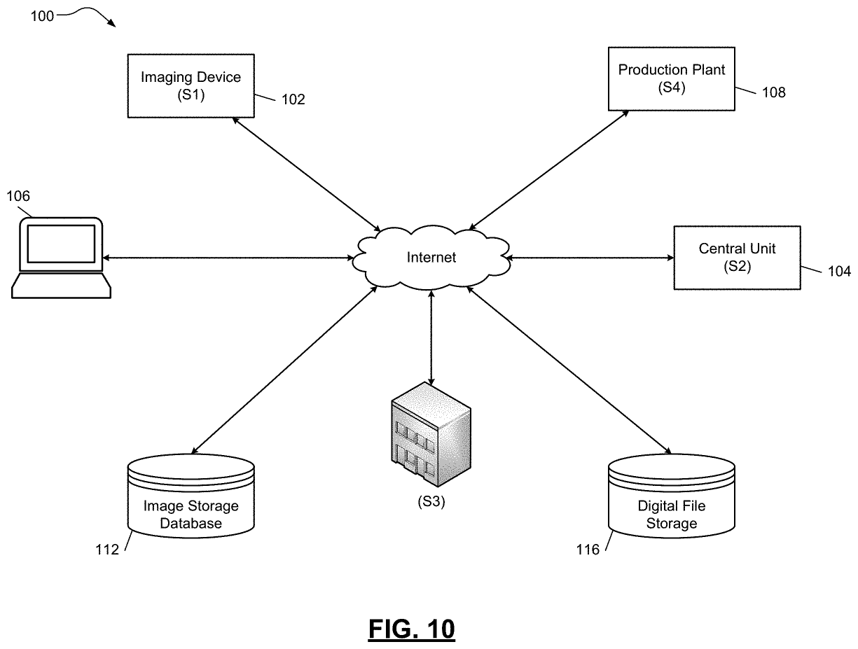

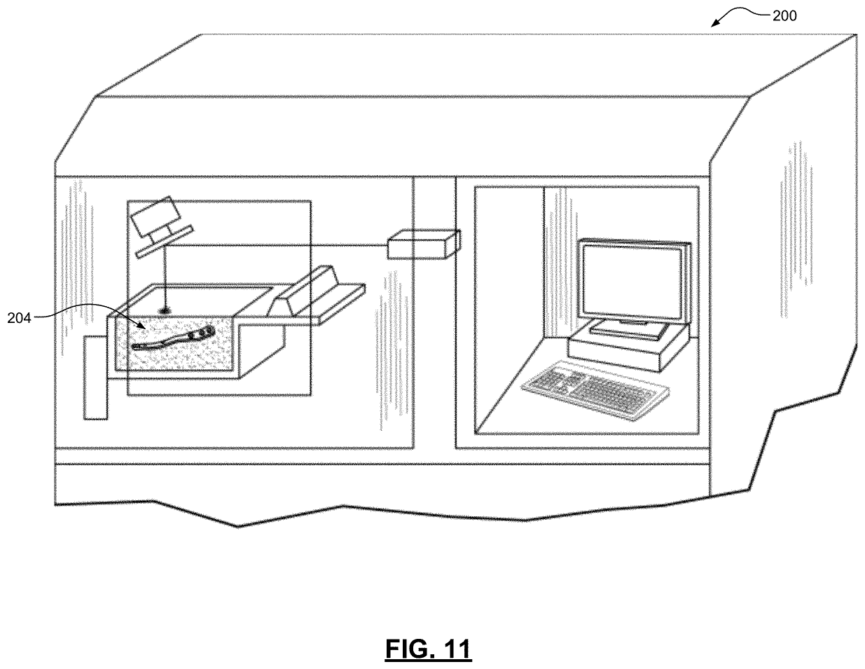

FIG. 9 is a view of a representation of the model of a perforated osteosynthesis plate 50 obtained. The model of the perforated osteosynthesis plate 40 can be sent to a device that makes it possible to manufacture an osteosynthesis plate from the model of the perforated osteosynthesis plate. The osteosynthesis plate that is produced is made of medical grade material.

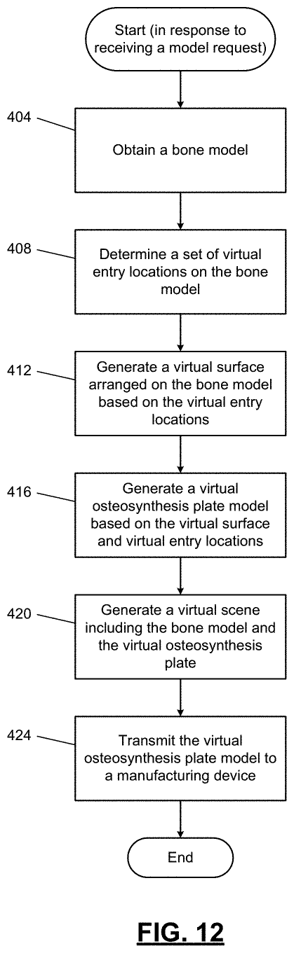

FIG. 10 is a schematic view of a system 100 according to the present disclosure. The system 100 according to the present disclosure can comprise an imaging device 102 situated in an imaging unit S1, for example in a hospital, a clinic, or a private facility dedicated to medical imaging. The three-dimensional data of the real bone can be sent to a central unit 104 in a location S2 remote from the location S1, for example in a cloud-type network infrastructure.

The user, typically a surgeon, can be situated in a third location S3—in his office, for example—and have a representation of a model of the bone generated by the central unit 104 can be displayed on an operator device 106. The device 108 making it possible to manufacture the osteosynthesis plate can be a 3D printer, for example, and be located in a production plant 108 at S4, in a location that is separate from the two previous location. The production plant 108 may include software stored in a memory and executed by a processor, for example, that is interfaced with that of a hospital or clinic for notifying the hospital planning software of the imminent availability of the osteosynthesis plate.

In various implementations, an image storage database 112 may store medical imaging files for a plurality of patients/users. The image storage database 112 may be accessible to the operator device 106, the imaging device 102 (for uploading of imaging files), the central unit 104, the production plant 108, etc. via the Internet. In various implementations, the imaging storage database 112 may be located on a local access network of one of the locations, for example, the production plant 108 or a hospital. Further, the system 100 includes a digital file storage 116, which may store generated digital files for production of a bone plate or surgical guide for a particular patient or user. The production plant 108 may obtain the digital files from the digital file storage 116 via the Internet to produce the bone plate or surgical guide.

Of course, the various features, forms, variants, and embodiments of the present disclosure can be combined with one another in various combinations as long as they are not incompatible or exclusive of one another. In particular, all of the variants and embodiments described above can be combined with one another.

FIG. 11 is an example additive manufacturing machine 200 creating a perforated osteosynthesis plate 204. In various implementations, the additive manufacturing machine 200 receives a perforated osteosynthesis plate model from a separate device that generates to model based on bone images. The additive manufacturing machine 200 can construct the perforated osteosynthesis plate 204 using the perforated osteosynthesis plate model implementing the methods described above.

FIG. 12 is a flowchart describing generation of a perforated osteosynthesis plate model. Control may be performed by a set of instructions stored in a memory of a device and operated by a processor of the device. For example, the instructions may be executed by the central unit 104. Control begins in response to receiving a model request from, for example, an operator or a surgeon to send generate the model and send the model for generation of the perforated osteosynthesis plate. At 404, control obtains a 3D bone model from, for example, data storage that stores imaging data for a plurality of users. In various implementations, the model request identifiers a user or patient and control obtain the 3D bone model corresponding to the user. In various implementations, the bone model is not 3D, for example, the bone model may include 2D images of the bone.

Control continues to 408 to determine a set of virtual entry locations on the bone model. Control proceeds to 412 to generate a virtual surface arranged on the bone model based on the virtual entry locations. As previously described the virtual surface may form a particular shape based on the virtual entry locations. Control proceeds to 416 to generate a virtual osteosynthesis plate model based on the virtual surface and the virtual entry locations, such as the virtual osteosynthesis plate model shown in FIG. 9.

Control proceeds to 420 to optionally generate a virtual scene including the bone model and the virtual osteosynthesis plate. In various implementations, control skips 420 and continues to 424. At 424, control transmits the virtual osteosynthesis plate model to a manufacturing device. In various implementations, at 424, control may transmit the virtual osteosynthesis model to a storage device, which may be accessible by the manufacturing device for production of an osteosynthesis plate.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A method of generating an osteosynthesis plate, comprising:
obtaining a bone model representing a bone generated from imaging data corresponding to the bone;
determining a set of virtual entry locations on the bone model;
constructing a virtual surface adjacent the bone model based on the bone model and the set of virtual entry locations;
generating an osteosynthesis plate model based on the virtual surface;
creating a virtual hole at each virtual entry location of the set of virtual entry locations along the osteosynthesis plate model; and
transmitting the osteosynthesis plate model to an osteosynthesis plate-manufacturing machine.

2. The method of claim 1 wherein the set of virtual entry locations indicate locations where a screw is inserted into the bone.

3. The method of claim 1 wherein the bone model is three-dimensional.

4. The method of claim 1 further comprising:
obtaining the imaging data corresponding to the bone from an imaging device.

5. The method of claim 1 wherein the virtual surface includes the set of virtual entry locations, a screw is one of multiple screws and the virtual hole is one of multiple holes.

6. The method of claim 1 wherein the osteosynthesis plate model includes a virtual face defined by the virtual surface and a thickness defined by an extrusion of the virtual face.

7. The method of claim 1 further comprising:
generating a virtual scene including the bone model, wherein the osteosynthesis plate model is pressed against the bone model and positioned such that a virtual face of the osteosynthesis plate model coincides with the virtual surface, and wherein
the osteosynthesis plate model generation and subsequent manufacturing of a custom osteosynthesis plate prevents a need for a surgeon to modify the osteosynthesis plate during surgery.

8. The method of claim 1 wherein the virtual surface is electronically determined from a virtual generatrix curve that passes successively through reference points, the reference points being determined as centers of the set of virtual entry locations and of at least two lateral virtual curves that are arranged on either side of the virtual generatrix curve.

9. The method of claim 8 wherein the reference points include longitudinal ends, with the virtual generatrix curve passing successively through one of the longitudinal ends, centers of the set of virtual entry locations, and another of the longitudinal ends.

10. The method of claim 8 wherein the virtual generatrix curve is a geodesic-type spline passing through the reference points.

11. The method of claim 8 wherein the at least two lateral virtual curves are geodesic-type splines that pass through images of the reference points, wherein the images are obtained through translation of a distance that is a function of a width of a screw at a greatest diameter perpendicular to an axis of the osteosynthesis plate model and tangent to the virtual surface of the bone model.

12. The method of claim 8 wherein the at least two lateral virtual curves are virtual curves that are spaced apart on each side of the virtual generatrix curve by a predetermined distance and projected on the virtual bone surface.

13. The method of claim 1 further comprising:
determining an intermediate osteosynthesis plate model by adding virtual fillets to the osteosynthesis plate model at longitudinal ends of the osteosynthesis plate model, wherein the determination of the osteosynthesis plate model is performed by modifying the intermediate osteosynthesis plate model.

14. The method of claim 1 wherein the osteosynthesis plate-manufacturing machine is an additive or subtractive manufacturing machine.

15. The method of claim 1 wherein the osteosynthesis plate that is manufactured is made of medical-grade materials.

16. The method of claim 1 wherein the osteosynthesis plate-manufacturing machine uses laser sintering or three-dimensional printing and is at a remote location from the modelling.

17. Computer software include instructions, stored in a non-transitory computer-readable memory, the software instructions comprising:
receiving a bone model of a bone generated from data representing the bone;
obtaining virtual entry locations on the bone model;
modelling an osteosynthesis plate model by:
determining a virtual surface on a face of the bone model based on the bone model and the virtual entry locations; and
drilling a virtual hole designed to receive a virtual screw at each virtual entry location along the osteosynthesis plate model; and
transmitting the osteosynthesis plate model to a remote osteosynthesis plate-manufacturer.

18. The instructions of claim 17 wherein the osteosynthesis plate model includes a virtual face defined by the virtual surface and a thickness defined by an extrusion of the virtual face.

19. The instructions of claim 17 further comprising:
generating a virtual scene including the bone model, wherein the osteosynthesis plate model is pressed against the bone model and positioned such that a virtual face of the osteosynthesis plate model coincides with the virtual surface.

20. The instructions of claim 17 wherein the virtual surface is determined from a virtual generatrix curve that passes successively through reference points, the reference points being determined as centers of the virtual entry locations and of at least two lateral virtual curves that are arranged on either side of the virtual generatrix curve.

21. The instructions of claim 20 wherein the reference points include longitudinal ends, with the virtual generatrix curve passing successively through one of the longitudinal ends, centers of the virtual entry locations, and another of the longitudinal ends.

22. A model generation system, comprising:
a graphical user interface;
at least one processor; and
a memory coupled to the at least one processor,
wherein the memory stores instructions executed by the at least one processor and
wherein the instructions include:
retrieving a bone model from imaging data corresponding to a bone;
displaying, via the display, the bone model;
receiving input, via the display, indicating a virtual entry location on the bone model;
constructing a virtual surface adjacent the bone model based on the bone model and the virtual entry location;
creating an osteosynthesis plate model based on the virtual surface;
drilling a virtual hole at the virtual entry location on the osteosynthesis plate model; and
transmitting the osteosynthesis plate model to a plate model storage.

23. The model generation system of claim 22 wherein the osteosynthesis plate model includes a virtual face defined by the virtual surface and a thickness defined by an extrusion of the virtual face.

24. The model generation system of claim 22 wherein the instructions include:
generating a virtual scene including the bone model, wherein the osteosynthesis plate model is pressed against the bone model and positioned such that a virtual face of the osteosynthesis plate model coincides with the virtual surface, and wherein
the osteosynthesis plate model generation and subsequent manufacturing of a custom osteosynthesis plate prevents a need for a surgeon to modify the plate during surgery.

25. The model generation system of claim 22 wherein the virtual surface is electronically determined from a virtual generatrix curve that passes successively through reference points, the reference points being determined as centers of the drill position and of at least two lateral virtual curves that are arranged on either side of the virtual generatrix curve.

26. The model generation system of claim 25 wherein the reference points include longitudinal ends, with the virtual generatrix curve passing successively through one of the longitudinal ends, centers of the drill position, and another of the longitudinal ends.
